(19) United States
(12) Patent Application Publication
(10) Pub. No.: US 2007/0260316 A1
(43) Pub. Date: Nov. 8, 2007

(54) Kit for an intervertebral implant and intervertebral implant

(75) Inventors: Susanne Schneid, Neu-Ulm (DE); Robert Schultz, Tuttlingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/799,332

(22) Filed: Apr. 30, 2007

Related U.S. Application Data
(63) Continuation of application No. PCT/EP05/12653, filed on Nov. 26, 2005.

(30) Foreign Application Priority Data
Dec. 9, 2004 (DE) 10 2004 059 298.5

(51) Int. Cl. A61F 2/44

(52) U.S. Cl. 623/17.15; 623/17.14

(57) ABSTRACT

In a kit for an intervertebral implant comprising an upper end plate and a lower end plate for placement on adjacent vertebral bodies, and bearing elements arranged between the end plates for mutually pivotally supporting these, the end plates carrying anchoring projections on their outer sides which face the vertebral bodies, to enable an anchoring of the end plates that is adapted to the anatomical conditions, it is proposed that the kit comprise end plates of a first type, which carry at least one anchoring projection configured as a fin or a rib extending parallel to the direction of insertion of the intervertebral implant, and end plates of a second type whose anchoring projections are different from fins or ribs extending parallel to the direction of insertion, and that the kit contain both end plates of the first type and end plates of the second type for each intervertebral implant. The invention also relates to an intervertebral implant assembled in this manner.

This application is a continuation of international application Number PCT/EP2005/012653 filed on Nov. 26, 2005.

The present disclosure relates to the subject matter disclosed in international application number PCT/EP2005/012653 of Nov. 26, 2005 and German application number 10 2004 059 298.5 of Dec. 9, 2004, which are incorporated herein by reference in their entirety and for all purposes.

### BACKGROUND OF THE INVENTION

The invention relates to a kit for an intervertebral implant comprising an upper end plate and a lower end plate for placement on adjacent vertebral bodies, and bearing elements arranged between the end plates for mutually pivotally supporting the end plates, the end plates carrying anchoring projections on their outer sides which face the vertebral bodies.

Intervertebral implants of this kind are used to replace damaged intervertebral discs between adjacent vertebral bodies. For anchoring the end plates, known intervertebral implants carry projections which engage the bone substance of the adjacent vertebral bodies, for example, in the form of spikes or in the form of ribs or fins, which extend parallel to the direction of insertion (EP 1 057 462 B1; WO 02/089701 A2; U.S. Pat. No. 6,740,118 B2; WO 01/01893 A1).

In intervertebral implants with fins extending in the direction of insertion, these are, in some cases, of such high construction as to enable good anchorage, but, it is then necessary to prepare the vertebral bodies accordingly, for example, by making a groove in the vertebral body, into which the fins or ribs can be pushed when inserting the intervertebral implant.

In particular, when inserting intervertebral implants on opposite sides of the same vertebral body, there is the danger that the vertebral body will be split by the grooves or notches. It is, therefore, often not possible to use such end plates with projections in the form of fins or ribs on both sides.

On the other hand, end plates which only have spikes or low transverse ribs for anchoring do not always provide the desired positional stabilization, as is the case, particularly when vertebral bodies with abnormal shapes are encountered, which do not lie over their entire surface against the end plates, but only partially.

The object of the invention is to propose a kit with which the operating surgeon has access to the respectively required conditions for anchoring the end plates to adjacent vertebral bodies.

### SUMMARY OF THE INVENTION

This object is accomplished, in accordance with the invention, with a kit of the kind described at the outset, in that it comprises end plates of a first type, which carry at least one anchoring projection configured as a fin or a rib extending parallel to the direction of insertion of the intervertebral implant, and end plates of a second type whose anchoring projections are different from fins or ribs extending parallel to the direction of insertion, and in that the kit contains both end plates of the first type and end plates of the second type for each intervertebral implant.

The operating surgeon, therefore, has the possibility of deciding during the operation whether to assemble the implant from end plates of the first type or end plates of the second type, and, where appropriate, end plates of the first type and end plates of the second type can also be used jointly on one and the same intervertebral implant. It is, for example, possible to provide both end plates with fins or ribs extending in the direction of insertion or to provide both end plates with anchoring projections which are different from these. It is also possible to produce an intervertebral implant in which the upper plate or the lower plate has a fin or a rib extending in the direction of insertion and the opposite plate is of different construction.

For example, the end plates of the second type can carry at least one rib extending transversely to the direction of insertion, it being advantageous for this rib to be lower than the rib or fin of the first type, which extends in the direction of insertion.

In another embodiment, it can be provided that the end plates of the second type carry at least one anchoring pin.

It is expedient for the kit to comprise both end plates of the first type and end plates of the second type for each size of end plate, so that, in addition, the operating surgeon can select from the kit end plates of different sizes, which selectively belong to the first type or the second type and which can be combined with one another either in the same size or in different sizes.

The kit thus contains differently sized end plates both of the first type and of the second type.

The invention also relates to an intervertebral implant comprising an upper end plate and a lower end plate for placement on adjacent vertebral bodies, and bearing elements arranged between the end plates for mutually pivotally supporting the end plates, the end plates carrying anchoring projections on their outer sides which face vertebral bodies.

To enable optimum adaptation of such an intervertebral implant to the anatomical conditions of the adjacent vertebral bodies, it is provided in accordance with the invention that the intervertebral implant comprises end plates of a first type, which carry at least one anchoring projection configured as a fin or a rib extending parallel to the direction of insertion of the intervertebral implant, and end plates of a second type whose anchoring projections are different from fins or ribs extending parallel to the direction of insertion.

The following description of preferred embodiments of the invention serves in conjunction with the drawings for a more detailed explanation.

### BRIEF DESCRIPTION OF THE DRAWINGS

FIG. 1 shows a side view of an intervertebral implant inserted between two vertebral bodies with a lower end plate of the first type and an upper end plate of the second type;

FIG. 2 shows the intervertebral implant of FIG. 1 prior to assembly; and

FIG. 3 shows a perspective view of an intervertebral implant with an upper end plate of the first type and a lower end plate of the second type prior to assembly.

### DETAILED DESCRIPTION OF THE INVENTION

The intervertebral implant 1 shown in the drawings is intended for insertion into the space between two adjacent vertebral bodies 2, 3. The intervertebral implant 1 comprises an upper end plate 4 and a lower end plate 5, which have contact surfaces on their outer sides for placement on the adjacent vertebral bodies 2, 3. The two end plates 4 and 5 are supported on each other for pivotal movement relative to each other. For this purpose a bearing body 6 is inserted between the lower end plate 5 and the upper end plate 4. The bearing body 6 has a spherical cap-shaped bearing surface 7 projecting upwardly, which engages a complementary bearing recess 8 on the upper end plate 4.

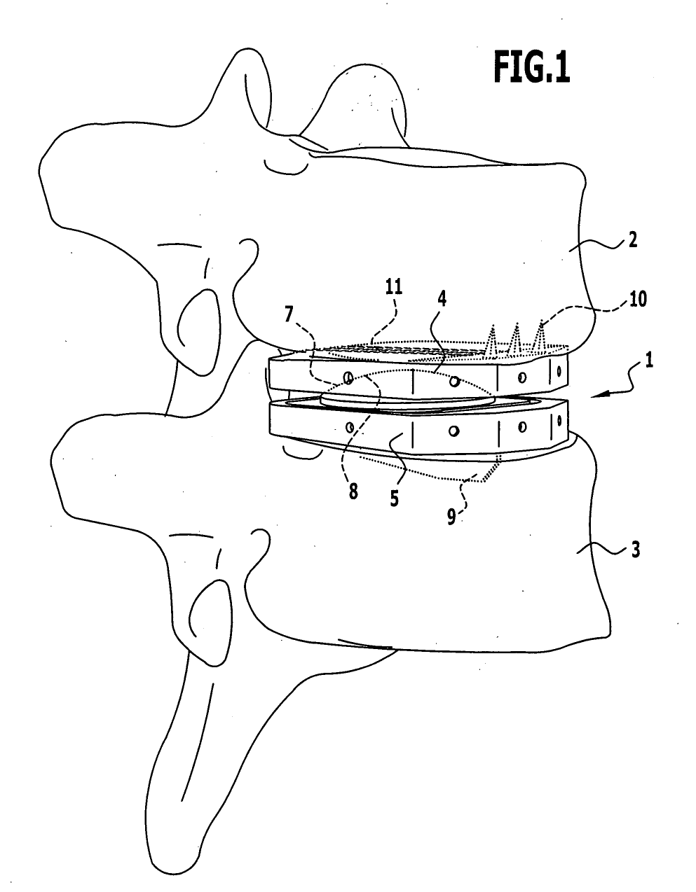

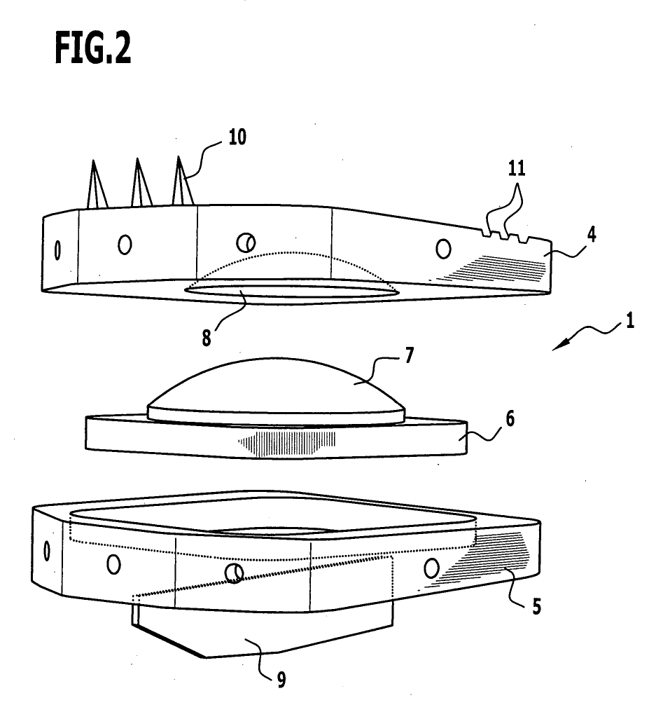

In the embodiment of FIGS. 1 and 2, the lower end plate carries on its outer side a central, perpendicularly upwardly projecting fin or rib 9, which extends over substantially the entire end plate 5 and parallel to a direction of insertion of the intervertebral implant 1. This rib 9 forms an anchoring projection which, when the intervertebral implant 1 is inserted, penetrates the adjacent vertebral body 3, which can be appropriately prepared for this, for example, by being provided with a corresponding groove.

Such an end plate having a fin or rib 9 extending in the direction of insertion will be referred to hereinbelow as end plate of the first type.

The opposite upper end plate 4 comprises in the embodiment shown in FIGS. 1 and 2 no such fin or rib 9, but instead anchoring projections of a different configuration, in the illustrated embodiment, in the form of upwardly projecting spikes 10 or in the form of low grooves or ribs 11 extending transversely to the direction of insertion. These ribs are produced by grooves being machined in the surface of the end plate.

Such an end plate which does not have an anchoring projection in the form of a rib or fin extending in the direction of insertion will be referred to hereinbelow as end plate of the second type. The anchoring projections herein may be of manifold configuration. What is essential is that these are not ribs or fins extending in the direction of insertion, but anchoring projections which also enable insertion of an end plate of the second type into the intervertebral space in a direction different from the normal direction of insertion, which usually extends in ventral-dorsal direction.

In the embodiment of FIG. 1, the lower end plate is an end plate of the first type, and the upper end plate is an end plate of the second type.

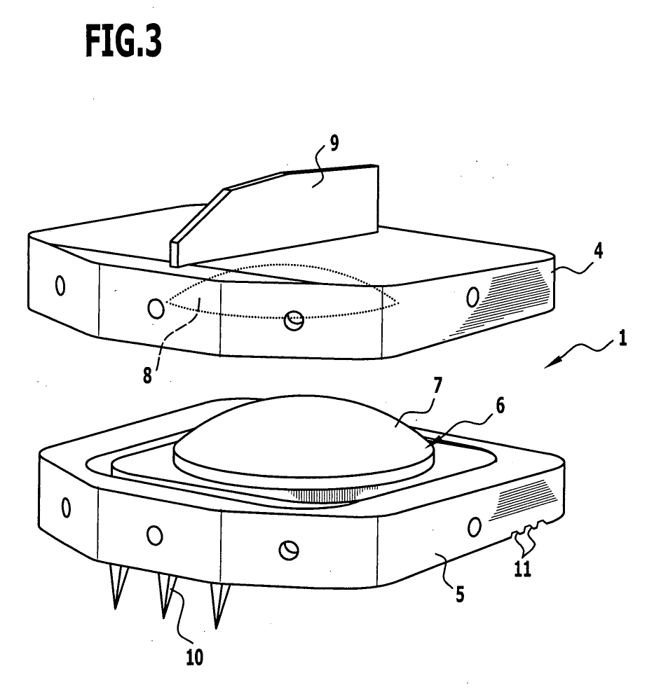

In the intervertebral implant of FIG. 3, the arrangement is chosen conversely, i.e., the upper end plate is an end plate of the first type and the lower end plate an end plate of the second type.

In a kit according to the invention, both upper end plates and lower end plates of the first and second types are provided for each size of intervertebral implant, so that the operating surgeon can optionally combine these end plates. He can, therefore, produce intervertebral implants in which both end plates have anchoring projections of the first type or both end plates have anchoring projections of the second type. It is also possible to produce intervertebral implants with combined end plates, as has been explained with reference to FIGS. 1 to 3.

It is also expedient for the kit to comprise end plates of different sizes, more specifically, both end plates of the first type and end plates of the second type for each size of end plate, so that intervertebral implants of different sizes with end plates of a different type can also be produced, more specifically, both intervertebral implants with end plates of the same size and intervertebral implants with end plates-of a different size.

1. Kit for an intervertebral implant comprising an upper end plate and a lower end plate for placement on adjacent vertebral bodies, and bearing elements arranged between the end plates for mutually pivotally supporting the end plates, said end plates carrying anchoring projections on their outer sides which face the vertebral bodies, said kit comprising end plates of a first type, which carry at least one anchoring projection configured as a fin or a rib extending parallel to the direction of insertion of the intervertebral implant, and end plates of a second type whose anchoring projections are different from fins or ribs extending parallel to the direction of insertion, and said kit containing both end plates of the first type and end plates of the second type for each intervertebral implant.

2. Kit in accordance with claim 1, wherein the end plates of the second type carry at least one anchoring pin.

3. Kit in accordance with claim 1, wherein the end plates of the second type carry at least one rib or groove extending transversely to the direction of insertion.

4. Kit in accordance with claim 3, wherein the grooves or ribs of the second type which extend transversely to the direction of insertion are lower than the ribs or fins of the first type.

5. Kit in accordance with claim 1, wherein said kit includes both end plates of the first type and end plates of the second type for each size of end plate.

6. Kit in accordance with claim 2, wherein said kit includes both end plates of the first type and end plates of the second type for each size of end plate.

7. Kit in accordance with claim 3, wherein said kit includes both end plates of the first type and end plates of the second type for each size of end plate.

8. Kit in accordance with claim 4, wherein said kit includes both end plates of the first type and end plates of the second type for each size of end plate.

9. Intervertebral implant comprising an upper end plate and a lower end plate for placement on adjacent vertebral bodies, and bearing elements arranged between the end plates for mutually pivotally supporting the end plates, said end plates carrying anchoring projections on their outer sides which face the vertebral bodies, said intervertebral implant including an end plate of a first type, which carries at least one anchoring projection configured as a fin or a rib extending parallel to the direction of insertion of the intervertebral implant, and an end plate of a second type whose anchoring projections are different from fins or ribs extending parallel to the direction of insertion.
